(19) United States
(12) Patent Application Publication
(10) Pub. No.: US 2011/0015433 A1
(43) Pub. Date: Jan. 20, 2011

(54) Simple preparation of trifluoromethionine and derivatives thereof

(75) Inventors: Norio SHIBATA, Nagoya (JP); Hiroyuki YASUI, Nagoya (JP)

Correspondence Address: OLIFF & BERRIDGE, PLC, P.O. BOX 320850, ALEXANDRIA, VA, 22320-4850, US

(73) Assignee: NATIONAL UNIVERSITY CORPORATION NAGOYA INSTITUTE OF TECHNOLOGY, NAGOYA-SHI (JP)

(21) Appl. No.: 12/801,717

(22) Filed: Jun. 22, 2010

(30) Foreign Application Priority Data
Jun. 26, 2009 (JP) 2009-151989

(51) Int. Cl. C07C 227/40

(52) U.S. Cl. 562/554

(57) ABSTRACT

Disclosed is a process for the simple preparation of trifluoromethionine, its analogs trifluoromethylcysteine, fluoroalkylhomocysteines, and fluoroalkylcysteines, and derivatives of them. These compounds are drug-candidate compounds or raw materials of drug-candidate compounds. Specifically, trifluoromethionine, trifluoromethylcysteine, a fluoroalkylhomocysteine, or a fluoroalkylcysteine is simply and conveniently prepared directly without passing through homocysteine or cysteine by adding metallic sodium to an optically active or racemic homocystine or cystine in liquid ammonia and further adding a fluoroalkyl iodide thereto under Birch reduction conditions.

### TECHNICAL FIELD

The present invention relates to a process for the simple preparation of trifluoromethionine, its analogs trifluoromethylcysteine, fluoroalkylhomocysteines, and fluoroalkylcysteines, and derivatives of them. These compounds are usable as drug-candidate compounds or as raw materials for drug-candidate compounds.

### BACKGROUND ART

There are annually forty-eight million patients suffering from entamebiasis around the world, in particular, in tropical developing countries, and the estimated number of deaths per year is about seventy thousand. In Japan, entamebiasis is designated as a category 5 infectious disease, and more than 700 cases have been reported annually.

Overview of entamebiasis: Entamoeba histolytica is an anaerobic or microaerophilic protozoan that is parasitic in the large intestinal tract. Infection to human is caused by ingesting a food or water contaminated with cysts. Excystation of the cysts occurs in the small intestine, and the cysts become trophozoites and reach the large intestine to form ulcerating pathogens on the mucosal surfaces of the large intestine including the rectum, sigmoid colon, cecum, and ascending colon. Not all infected subjects exhibit symptoms of the disease, and only 5% to 10% of the infected subjects are considered to exhibit symptoms of the disease. Subjects exhibiting symptoms of the disease show dysenteric symptoms such as mucous and bloody stool, diarrhea, tenesmus, aerenterectasia, and lower abdominal pain during defecation. In typical cases, patients pass mucous and bloody stool, and exacerbation and remission are repeated at intervals of several days to several weeks. In exacerbation cases, enterobrosia is caused. Extraintestinal lesions are observed in about 5% of patients exhibiting colitis symptoms. In particular, abscesses are formed in organs or tissues such as liver, lung, brain, and skin. Of those, liver abscesses are most frequently caused and are accompanied by fever with temperatures of 38° C. to 40° C., hypochondrial pain, nausea, vomiting, weight loss, night sweat, or generalized fatigability. When abscesses burst, lesions are formed in the peritoneum, pleura, and epicardium, resulting in severe symptoms. Trophozoites are encysted in the large intestine and discharged to feces, which are orally ingested by another person, thereby infection is established.

Entamebiasis treatment: In general, treatment of entamebiasis is performed by oral administration of metronidazole (product name: Flagyl), which has a high therapeutic effect on symptomatic persons. However, metronidazole is absorbed well from the digestive tract but has a low killing effect on cysts in the intestinal tract, and it is not effective for group treatment of cyst carriers. In order to treat the carriers, metronidazole is used together with diloxanide furoate, which is absorbed from the digestive tract at a low efficiency. However, the killing effect on cysts is insufficient in some cases. Another problem of metronidazole is to readily cause in vitro resistance. In view of the actual state of appearance of resistant strains in another protozoan such as Plasmodium, it is only a matter of time before an Entamoeba histolytica strain resistant to metronidazole appears. In the case of Trichomonad that is an anaerobic protozoan like Entamoeba histolytica, a clinical strain resistant to metronidazole has been reported. Therefore, it is necessary to synthesize a novel compound having the effect of killing Entamoeba histolytica. Trifluoromethionine (compound A shown below) has been reported to have protozoicidal activity on anaerobic protozoans such as Entamoeba histolytica and Trichomonas vaginalis (Non Patent Literature 1 or 2) . The pesticidal activity of trifluoromethionine on the protozoans depends on an enzyme that is present specifically in protozoan and called methionine gamma-lyase. Methionine gamma-lyase is an enzyme involved in decomposition of sulfur-containing amino acids and is not present in mammals, and therefore, the compound has selective effects for protozoans.

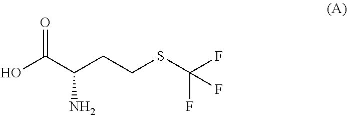

The present inventors have further found that derivatives of the compound A, i.e., trifluoromethionine derivatives show higher proliferation inhibiting activities on Entamoeba histolytica (Patent Literature 1).

There are several production processes of trifluoromethionine as disclosed typically in Non Patent Literatures 3, 4, 5, and 6. According to these processes, trifluoromethionine is prepared from starting material homocysteine which is in turn prepared from homocystine or methionine.

Citation List

### Patent Literature

PTL 1: PCT International Publication Number WO/2007077876 Non Patent Literature

NPL 1: G. H. Coombs, J. C. Mottram, Antimicrob. Agents Chemother 2001, 45, 1743

NPL 2 : M. Tokoro et al . , J. Biol. Chem. 2003, 278, 42717-42727

NPL 3: R. L. Dannley, R. G., Taborsky, J. Org. Chem. 1957, 22, 55

NPL4: M. E. Houston, J. F. Honek, J. Chem. Soc. Chem. Commun. 1989, 761.

NPL 5: V. Soloshonok, V. Kukhar, Y. Pustovit, V. Nazaretian, Synlett 1992, 657.

NPL 6: Tadashi Shiraiwa, Chem. Pharm. Bull. 2002, 50, 1081.

### SUMMARY OF INVENTION

Technical Problem

Though being potential preparation processes, the customary preparation processes of trifluoromethionine via homocysteine are insufficient in yield and purification procedure of homocysteine. Under these circumstances, an object of the present invention is to provide a process for the simple preparation of trifluoromethionine in a high yield directly without isolation and purification of homocysteine. Such process according to the present invention can also be applied to the simple preparation of trifluoromethylcysteine, fluoroalkylhomocysteines, and fluoroalkylmethylcysteines.
Solution to Problem

Specifically, to achieve the above object, the present invention provides, in a first aspect, a process for the simple preparation of a fluoroalkylhomosysteine or fluoroalkylcysteine, which process includes the steps of dissolving homocystine or cystine in liquid ammonia; adding metallic sodium thereto while avoiding the evaporation of ammonia; further adding a fluoroalkyl iodide thereto under Birch reduction conditions to give a reaction mixture; evaporating ammonia from the reaction mixture to give a solid as a residue; and purifying the solid.

The present invention further provides, in a second aspect, a process for the simple preparation of an optically-active or racemic compound. This process includes the steps of dissolving an optically-active or racemic homocystine or cystine as a starting material in liquid ammonia; adding metallic sodium thereto while avoiding the evaporation of ammonia; further adding a fluoroalkyl iodide thereto under Birch reduction conditions to give a reaction mixture; evaporating ammonia from the reaction mixture to give a solid as a residue; and purifying the solid, in which the optically-active or racemic compound is represented by following General Formula (I) :

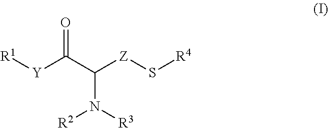

wherein Y represents oxygen or NH; Z represents —(CH₂)ₘ—, wherein m denotes an integer of 1 or 2; R¹ represents one of following (i) to (v) , or amino acid or peptide; R² and R³ each independently represent hydrogen, formyl group, acetyl group, butyloxycarbonyl (Boc) group, amino acid, or peptide; and R⁴ represents fluoroalkyl group of formula: —(CH₂)ₖ— (CF₂)₁—F, wherein k and l each denote an integer of 0 to 5 and wherein the total of k and l is from 1 to 5:

(i) hydrogen;

(ii)

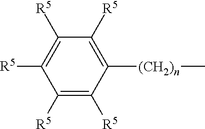

wherein n denotes an integer of 0 to 5; and R⁵s each independently represent hydrogen, halogen, alkoxy having 1 to 5 carbon atoms, or alkyl having 1 to 5 carbon atoms;

(iii)

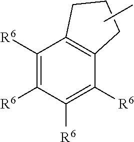

wherein R⁶s each independently represent hydrogen, halogen, alkoxy having 1 to 5 carbon atoms, or alkyl having 1 to 5 carbon atoms;

(iv) alkyl having 1 to 5 carbon atoms; and

(v) hydroxyalkyl having 1 to 5 carbon atoms.

In a preferred embodiment of the process, R¹ and R² and R³ are hydrogens; Y is oxygen; Z is —CH₂— or —(CH₂)₂—; and R⁴ is CF₃ or CF₂CF₃ in the optically-active or racemic compound of General Formula (I) prepared from the optically-active or racemic homocystine or cystine as the starting material.

In another preferred embodiment of the process, the starting material is one selected from the group consisting of L-homocystine, L-cystine, and N-Boc-L-homocystine as an L-homocystine derivative, and the resulting optically-active or racemic compound is one selected from the group consisting of the following compounds :

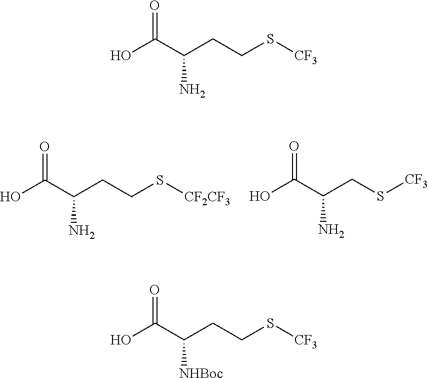

The process according to the present invention enables the simple preparation (synthesis) of trifluoromethionine directly without isolation of homocysteine, by adding trifluoromethyl iodide to homocystine in liquid ammonia in the presence of metallic sodium under Birch reduction conditions. Such a process can be adopted to the preparation of a variety of fluoroalkylhomocysteines while varying the type of the fluoroalkyl iodide to be added. Typically the reaction of cystine with trifluoromethyl iodide gives trifluoromethylcysteine; and the reaction of cystine with a fluoroalkyl iodide gives a corresponding fluoroalkylcysteine.

Specifically, the present invention provides a process for the simple preparation of a compound represented by following General Formula (I) . This process adopts an optically-active or racemic homocystine or cystine as a starting material and is for the simple preparation of an optically-active or racemic compound of General Formula (I) :

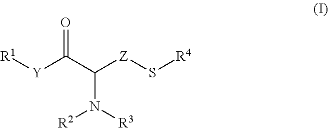

wherein Y represents oxygen or NH; Z represents —(CH₂)ₘ—, wherein m denotes an integer of 1 or 2; R¹ represents one of following (i) to (v) , or amino acid or peptide; R² and R³ each independently represent hydrogen, formyl group, acetyl group, butyloxycarbonyl (Boc) group, amino acid, or peptide; and R⁴ represents fluoroalkyl group of formula: —(CH₂)ₖ— (CF₂)₁—F, wherein k and l each denote an integer of 0 to 5 and wherein the total of k and l is from 1 to 5:

(i) hydrogen;

(ii)

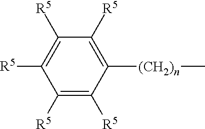

wherein n denotes an integer of 0 to 5; and R⁵s each independently represent hydrogen, halogen, alkoxy having 1 to 5 carbon atoms, or alkyl having 1 to 5 carbon atoms;

(iii)

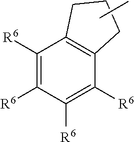

wherein R⁶s each independently represent hydrogen, halogen, alkoxy having 1 to 5 carbon atoms, or alkyl having 1 to 5 carbon atoms;
(iv) alkyl having 1 to 5 carbon atoms; and
(v) hydroxyalkyl having 1 to 5 carbon atoms.

### BRIEF DESCRIPTION OF DRAWINGS

FIG. 1 is the general formula of compounds prepared by the simple preparation process according to the present invention.

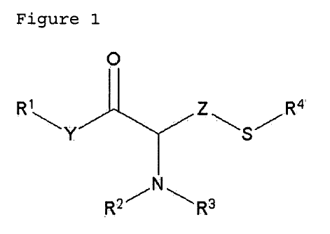

### DESCRIPTION OF EMBODIMENTS

The present invention will be illustrated in detail below. Initially, homocystine or cystine is dissolved in liquid ammonia. Homocystine or cystine to be used can be in D-form, L-form, or DL-form. It has been demonstrated that the steric configuration of the compound is maintained after the reaction. In this step, water should be thoroughly removed from the reaction system so as to avoid the contamination of homocysteine or cysteine into the product. The reaction should be performed while cooling the mixture (solution) to a low temperature of −35° C. to −80° C. so as to avoid the evaporation of ammonia. The mixture is preferably cooled to around −80° C., because Birch reduction is an exothermic reaction. Next, metallic sodium is gradually added to the mixture while keeping the mixture at a temperature below —35° C. The metallic sodium may be added in excess but is preferably added in an amount of 4.2 equivalents to homocystine or cystine. Next, trifluoromethyl iodide in excess, preferably 2.5 equivalents, is gradually added to the mixture. After stirring for 20 minutes, ammonia is gradually evaporated to give a solid, and the solid is purified through ion-exchange column chromatography to give the target trifluoromethionine or trifluoromethylcysteine. The process can also give a corresponding fluoroalkylhomocysteine or fluoroalkylcysteine by varying the fluoroalkyl iodide to be added.

Compounds which can be prepared according to the present invention have the following structural formulae.

Specifically, the compounds which are prepared from an optically-active or racemic homocystine or cystine are optically-active or racemic compounds represented by following General Formula (I):

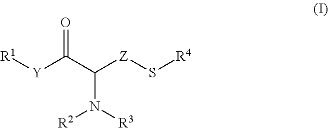

wherein Y represents oxygen or NH; Z represents —(CH₂)ₘ—, wherein m denotes an integer of 1 or 2; R¹ represents one of following (i) to (v) , or amino acid or peptide; R² and R³ each independently represent hydrogen, formyl group, acetyl group, butyloxycarbonyl (Boc) group, amino acid, or peptide; and R⁴ represents fluoroalkyl group of formula: —(CH₂)ₖ— (CF₂)₁—F, wherein k and l each denote an integer of 0 to 5 and wherein the total of k and 1 is from 1 to 5:

(i) hydrogen

(ii)

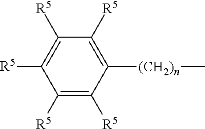

wherein n denotes an integer of 0 to 5; and R⁵s each independently represent hydrogen, halogen, alkoxy having 1 to 5 carbon atoms, or alkyl having 1 to 5 carbon atoms;

(iii)

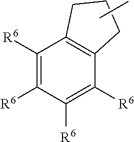

wherein R⁶s each independently represent hydrogen, halogen, alkoxy having 1 to 5 carbon atoms, or alkyl having 1 to 5 carbon atoms;

(iv) alkyl having 1 to 5 carbon atoms; and

(v) hydroxyalkyl having 1 to 5 carbon atoms.

The present invention will be illustrated in further detail with reference to several embodiments as working examples below. It should be noted, however, that these examples are never construed to limit the scope of the present invention.

### FIRST EMBODIMENT

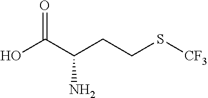

In a 500-ml three-necked flask equipped with a liquid-ammonia cooling device, a glass stirring bar, a thermometer, and a septum rubber cap was placed 10.0 g (37.3 mmol) of L-homocystine, followed by thorough purging of the flask with nitrogen. After cooling the flask to —78° C., ammonia which had been dried by passing through a KOH tube was liquefied in the liquid-ammonia cooling device cooled on a dry ice-acetone bath, and the liquid ammonia was added in a volume of about 100 ml to L-homocystine, followed by stirring. Next, 3.58 g (156 mmol) of metallic sodium was gradually added while avoiding rise of the temperature of the mixture above −35° C. The solution (mixture) became dark blue in this step. Next, 18.2 g (93.3 mmol) of trifluoromethyl iodide weighed using a balloon was added, the mixture was stirred on a bath at −78° C. for 20 minutes, from which ammonia was gradually evaporated, the residue was dissolved in ultrapure water, and the solution was placed on an ion exchange resin DOWEX-50X8 which had been treated with hydrochloric acid. After passing a sufficient amount of ultrapure water, elution with a 2% aqueous ammonia solution was performed, and thereby yielded 14.1 g of target L-trifluoromethionine in a yield of 93%.
¹H-NMR (D₂O, 200 MHz) δ: 2.07-2.37 (m, 2H) , 3.01 (t, 2H, J=7.6 Hz), 4.03 (t, 1H, J=6.6 Hz)
¹⁹F-NMR (D₂O, 188 MHz) δ: −41.3 (s)

### SECOND EMBODIMENT

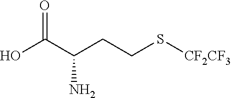

In a 200-ml three-necked flask equipped with a liquid-ammonia cooling device, a glass stirring bar, a thermometer, and a septum rubber cap was placed 1.00 g (3.73 mmol) of L-homocystine, followed by thorough purging of the flask with nitrogen. After cooling the flask to −78° C., ammonia which had been dried by passing through a KOH tube was liquefied in the liquid-ammonia cooling device cooled on a dry ice-acetone bath, and the liquid ammonia was added in a volume of about 50 ml to L-homocystine, followed by stirring. Next, 358 mg (15.6 mmol) of metallic sodium was gradually added while avoiding rise of the temperature of the mixture above −35° C. The solution (mixture) became dark blue in this step. Next, 2.29 g (9.33 mmol) of pentafluoroethyl iodide weighed using a balloon was added, the mixture was stirred on a bath at −78° C. for 20 minutes, from which ammonia was gradually evaporated, the resulting residue was dissolved in ultrapure water to give a solution, the solution was placed on the ion exchange resin DOWEX-50X8 which had been treated with hydrochloric acid. After passing a sufficient amount of ultrapure water, elution with a 2% aqueous ammonia solution was performed, and thereby yielded 1.27 g of target L-pentafluoroethylhomocysteine (L-pentafluoroethionine) in a yield of 67%.
¹H-NMR (D₂O, 200 MHz) δ: 2.03-2.34 (m, 2H) , 3.01 (t, 2H, J=7.6 Hz), 4.04 (t, 1H, J=6.6 Hz)
¹⁹F-NMR (D₂O, 188 MHz) δ: −93.8 (s), −85.3 (s)

### THIRD EMBODIMENT

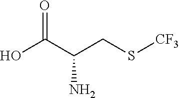

In a 200-ml three-necked flask equipped with a liquid-ammonia cooling device, a glass stirring bar, a thermometer, and a septum rubber cap was placed 0.961 g (4.00 mmol) of L-cystine, followed by thorough purging of the flask with nitrogen. After cooling the flask to −78° C., ammonia which had been dried by passing through a KOH tube was liquefied in the liquid-ammonia cooling device cooled on a dry ice-acetone bath, and the liquid ammonia was added in a volume of about 30 ml to L-cystine, followed by stirring. Next, 385 mg (16.8 mmol) of metallic sodium was gradually added while avoiding rise of the temperature of the mixture above −35° C. The solution (mixture) became dark blue in this step. Next, 1.96 g (10.0 mmol) of trifluoromethyl iodide weighed using a balloon was added, the mixture was stirred on a bath at −78° C. for 20 minutes, from which ammonia was gradually evaporated, the residue was dissolved in ultrapure water to give a solution, and the solution was placed on an ion exchange resin DOWEX-50X8 which had been treated with hydrochloric acid. After passing a sufficient amount of ultrapure water, elution with a 2% aqueous ammonia solution was performed, and thereby yielded 0.983 g of target L-trifluoromethylcysteine in a yield of 65%.
¹H-NMR (D₂O, 200 MHz) δ: 3.21 (dd, 1H, J=7.6 Hz, 15.2 Hz) , 3.39 (dd, 1H, J=7.6 Hz, 15.2 Hz), 3.86 (t, 1H, J=7.6 Hz)
¹⁹F-NMR (D₂O, 188 MHz) δ: −41.4 (s)

### FOURTH EMBODIMENT

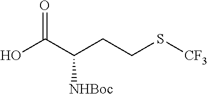

In a 200-ml three-necked flask equipped with a liquid-ammonia cooling device, a glass stirring bar, a thermometer, and a septum rubber cap was placed 1.87 g (4.00 mmol) of N-Boc-L-homocystine, followed by thorough purging of the flask with nitrogen. After cooling the flask to −78° C., ammonia which had been dried by passing through a KOH tube was liquefied in the liquid-ammonia cooling device cooled on a dry ice-acetone bath, and the liquid ammonia was added in a volume of about 30 ml to N-Boc-L-homocystine , followed by stirring. Next, 385 mg (16.8 mmol) of metallic sodium was gradually added while avoiding rise of the temperature of the mixture above −35° C. The solution (mixture) became dark blue in this step. Next, 1.96 g (10.0 mmol) of trifluoromethyl iodide weighed using a balloon was added, the mixture was stirred on a bath at −78° C. for 20 minutes, from which ammonia was gradually evaporated, the residue was dissolved in ultrapure water to give a solution, the solution was adjusted with a 1 N aqueous citric acid solution to have a pH of 3.0 and extracted three portions of 30 mL of ethyl acetate. The organic phase was washed with saturated aqueous sodium chloride solution and dried over anhydrous sodium sulfate. After concentrating on an evaporator, the solvent was thoroughly removed in vacuo, to give 1.70 g of target N-Boc-L-trifluoromethionine in a yield of 70%.
¹H-NMR (CDCl₃, 200 MHz) δ: 1.46 (s, 9H) , 2.05-2.12 (m, 1H), 2.31-2.34 (m, 1H), 2.98 (t, 2H, J=7.6 Hz), 4.30-4.42 (br, 1H), 8.45 (br, 1H)
¹⁹F-NMR (CDCl₃, 188 MHz) δ: −42.0 (s)

Compounds prepared by the process according to the present invention can be used as therapeutic drugs or prophylactic drugs for infectious diseases caused by protozoans or bacteria.

1. A process for the simple preparation of a fluoroalkylhomosysteine or fluoroalkylcysteine, the process comprising the steps of:
dissolving homocystine or cystine in liquid ammonia;
adding metallic sodium thereto while avoiding the evaporation of ammonia;
further adding a fluoroalkyl iodide thereto under Birch reduction conditions to give a reaction mixture;
evaporating ammonia from the reaction mixture to give a solid as a residue; and
purifying the solid.

2. A process for the simple preparation of an optically-active or racemic compound, the process comprising the steps of:
dissolving an optically-active or racemic homocystine or cystine as a starting material in liquid ammonia;
adding metallic sodium thereto while avoiding the evaporation of ammonia;
further adding a fluoroalkyl iodide thereto under Birch reduction conditions to give a reaction mixture;
evaporating ammonia from the reaction mixture to give a solid as a residue; and
purifying the solid,
the optically-active or racemic compound represented by following General Formula (I): 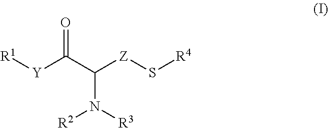
wherein Y represents oxygen or NH; Z represents —(CH₂)ₘ—, wherein m denotes an integer of 1 or 2; R¹ represents one of following (i) to (v) , or amino acid or peptide; R² and R³ each independently represent hydrogen, formyl group, acetyl group, butyloxycarbonyl (Boc) group, amino acid, or peptide; and R⁴ represents fluoroalkyl group of formula: —(CH₂)ₖ— (CF₂)₁—F, wherein k and l each denote an integer of 0 to 5 and wherein the total of k and l is from 1 to 5:
(i) hydrogen;
(ii) 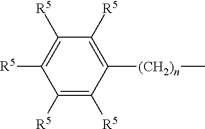
wherein n denotes an integer of 0 to 5; and R⁵s each independently represent hydrogen, halogen, alkoxy having 1 to 5 carbon atoms, or alkyl having 1 to 5 carbon atoms;
(iii) 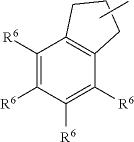
wherein R⁶s each independently represent hydrogen, halogen, alkoxy having 1 to 5 carbon atoms, or alkyl having 1 to 5 carbon atoms;
(iv) alkyl having 1 to 5 carbon atoms; and
(v) hydroxyalkyl having 1 to 5 carbon atoms.

3. The process according to claim 2, wherein R¹ and R² and R³ are hydrogens; Y is oxygen; Z is —CH₂— or —(CH₂)₂—; and R⁴ is CF₃ or CF₂CF₃ in the optically-active or racemic compound of General Formula (I) prepared from the optically-active or racemic homocystine or cystine as the starting material.

4. The process according to claim 2, wherein the starting material is one selected from the group consisting of L-homocystine, L-cystine, and N-Boc-L-homocystine as an L-homocystine derivative, and wherein the resulting optically-active or racemic compound is one selected from the group consisting of the following compounds: [chemical structure]
